SEQUENCE LISTING (8 sequences)

SEQ ID NO: 1 - protein, 23 aa, Influenza A
SLLTEVETPIRNEWGCRCNGSSD

SEQ ID NO: 2 - protein, 23 aa, Influenza A
SLLTEVETLTRNGWGCRCSDSSD

SEQ ID NO: 3 - protein, 23 aa, Influenza A
SLLTEVETPTRNGWECKCSDSSD

SEQ ID NO: 4 - protein, 23 aa, Influenza A
SLLTEVETPIKNEWECRCNDSSD

SEQ ID NO: 5 - DNA, 23 bp, Influenza A
aagaccaatcctgtcacctctga

SEQ ID NO: 6 - DNA, 22 bp, Influenza A
caaagcgtctacgctgcagtcc

SEQ ID NO: 7 - DNA, 21 bp, Influenza A
tttgtgttcacgctcaccgtt

SEQ ID NO: 8 - protein, 9 aa, Artificial Sequence
  peptide tetramer
ASNENTETM

(19) United States
(12) Patent Application Publication
(10) Pub. No.: US 2013/0209512 A1
(43) Pub. Date: Aug. 15, 2013

(54) UNIVERSAL INFLUENZA A VACCINES

(71) Applicants: Hildegund C. J. Ertl, Philadelphia, PA (US); Dongming Zhou, Philadelphia, PA (US)

(72) Inventors: Hildegund C. J. Ertl, Philadelphia, PA (US); Dongming Zhou, Philadelphia, PA (US)

(21) Appl. No.: 13/817,100

(22) PCT Filed: Aug. 16, 2011
(86) PCT No.: PCT/US11/47900

§ 371 Date: Apr. 24, 2013

Related U.S. Application Data
(60) Provisional application No. 61/374,024, filed on Aug. 16, 2010.
(60) Provisional application No. 61/487,004, filed on May 17, 2011.

(51) Int. Cl. C07K 14/11

(52) U.S. Cl. CPC C07K 14/11
USPC 424/209.1; 530/358; 536/23.4; 435/320.1

(57) ABSTRACT

Universal flu vaccines are disclosed. The vaccines induce broad and sustained protection against a wide range of influenza A viruses, reduce the need for annual vaccination campaigns with vaccines based upon viral strains predicted to be the predominant circulating strains, and ameliorate the threat of future pandemics that can potentially kill millions.

This application claims the benefit of Ser. No. 61/374,024 filed on Aug. 16, 2010 and Ser. No. 61/487,004 filed on May 17, 2011.

Each reference cited in this disclosure is incorporated herein by reference in its entirety.

This invention was made with government support under HHSN266200500030C awarded by National Institutes of Health. The government has certain rights in the invention.

### BACKGROUND OF THE INVENTION

Influenza A viruses are negative-sense, single-stranded, segmented RNA viruses, which contain 8 RNA segments, encoding for 11 proteins (HA, NA, NP, M1, M2, NS1, NEP/NS2, PA, PB1, PB1-F2, PB2). Matrix protein 2 (M2) is a tetrameric transmembrane protein of influenza A virus. Its ectodomain (M2e) shows conservation among human influenza A virus strains. M2e-specific antibodies, although not neutralizing, reduce in animals the severity of infection with a wide range of influenza A virus strains.^{3,4} Influenza A nucleoprotein (NP), the major protein component of ribonucleoprotein (RNP) complexes is also relatively conserved making it an attractive candidate for a universal flu vaccine. Although the NP protein induces an antibody response, the role of such antibodies in providing protection remains controversial.^{5,6} The NP induces a vigorous CD8⁺ T cell response both in mice and men^{7,8} that, as epidemiological studies suggest, may contribute to resistance against severe disease following influenza A virus infection.⁹

Influenza vaccines based on M2e, NP or both have been tested extensively in animal models, where they have shown sufficient promise that some of them advanced to clinical trials.^{3,4, 10-14} However, results from efficacy trials, which have commonly only shown limited efficacy even for licensed vaccines,^{15, 16} are not yet available.

Various strategies have been tested to develop a universal flu vaccine. Most of these strategies focused on vaccines expressing the M1, M2 or NP of influenza virus^{3, 4, 10-14, 22} which are relatively conserved between different viral strains. Both the M1 and the NP are dominant targets CD8⁺ T cells in humans while the ectodomain of M2 binds non-neutralizing antibodies that provide some, albeit limited, protection. Although epidemiological evidence supports the development of CD8⁺ cell vaccines to influenza. A viruses,⁹ in animal models, the effectiveness of CD8⁺ cells in protecting against influenza A viruses remains controversial; while some investigators reported induction of protection,²² others reported lack of efficacy²³ or even exacerbation of disease following viral challenge.²⁴ In our hands vaccines that induce very high frequencies of NP-specific CD8⁺ T cells such as Ad vectors expressing the NP provided only marginal protection.

M2e is poorly immunogenic during a natural influenza A virus infection due to the paucity of its expression on virions.²⁵ Humans develop M2e-specific antibodies following infection but titers are low and not sustained.²⁶ Several studies conducted in mice and ferrets have shown that M2e-specific antibodies can restrict subsequent virus replication and reduce morbidity and mortality to a broad range of influenza A virus strains.^{3, 4} Although M2e is relatively conserved variability between different strains has been described²⁷ and vaccines that express only one version of M2e may thus lack efficacy against strains with M2e mutations. By the same token it was shown that antibodies to M2e select for escape mutants.²⁸ A number of vaccine platforms have been tested^{3, 4,10-14} and several of those such as the M2e-hepatitis B core protein vaccine subsequently underwent early clinical testing which confirmed their safety and immunogenicity.³

### BRIEF DESCRIPTION OF THE FIGURES

FIGS. 1A-C, M2e(3)-NP fusion protein. FIG. 1A, amino acid sequences of three M2e in the construct and of A/Fort Monmouth/1/47 virus. H1N1-M2e, SEQ ID NO:1; H5N1-M2e, SEQ ID NO:2; H7N2-M2e, SEQ ID NO:3; H1N1-M2e, SEQ ID NO:4, FIG. 1B, schematic representation of the chimeric M2e(3)-NP gene. FIG. 1C, expression of M2e(3)-NP protein by different vectors in infected cells in comparison to β-actin, * Same substrain.

FIGS. 2A-B, humoral responses in C57Bl/6 mice or ICR mice. FIG. 2A, three cell lines expressing M2 of H1N1, H5N1 or H7N2 were used in cellular ELISAs to measure M2e-specific antibody titers in sera of C57Bl/6 mice (n=10) harvested 3 months after priming or 5 weeks after boosting. FIG. 2B, M2e-specific antibody titers in sera of ICR mice (n=10) harvested 3 months after priming or 5 weeks after boosting. *p<0.05; **p<0.01.

FIGS. 3A-B, NP-specific CD8⁺ cell responses in C57Bl/6 mice. FIG. 3A, intracellular INF-γ staining of NP-specific CD8⁺ T cells was carried out on PBMCs from mice (n=10) at weeks 2, 5, 8, 10 and 12, after vaccination. Prime-boosted mice (filled circles), primed mice (empty squares). FIG. 3B, Frequencies of NP-specific CD8⁺ T cells in lung, blood and spleen tested 4 months after priming or 2 months after boosting by tetramer staining. *p<0.05; **p<0.01.

FIGS. 4A-F, protection against challenge. FIG. 4A, C57Bl/6 mice were challenged with 10LD₅₀ of A/PR/8 virus 4 months after priming or 2 months after boosting. Five days later lung virus titers were determined. Graph shows titers of viral genomes (vgs) per gram of tissue of individual mice (open circles) or as geometric mean titers (X). Prime-boost mice (n=15) vs. naïve mice (n=10), p=0.0003, primed mice (n=10) vs. naïve mice (n=10), p=0.2. FIG. 4B, weight loss after challenge with 10LD₅₀ A/PR/8; vaccinated mice (filled circles), naïve mice (empty circles). FIG. 4C, weight loss after challenge with 10LD₅₀ A/Fort Monmouth virus; vaccinated mice (filled circles), naïve mice (empty circles), FIG. 4D, survival curve of the mice after challenge with A/PR/8; vaccinated (n=9) versus naïve mice (n=9), p=0.0002. FIG. 4E, survival curve of the mice after challenge with A/Fort Monmouth virus; vaccinated (n=9) versus naïve mice (n=9), p=0.0002. FIG. 4F, stained lung sections from a mouse that received the prime-boost and from a naïve mouse both harvested 5 days after challenge with 10LD₅₀ A/PR/8 virus. HE-stained sections are shown at 100× magnification.

FIGS. 5A-E, protection of ICR and BALB/c mice. FIG. 5A, ICR mice were challenged with 10LD₅₀ A/PR/8 4 months after priming or two months after the boost. Five clays after challenge, lung virus titers were determined. Graph shows titers of vgs per gram of tissue of individual mice (open circles) or as geometric mean titers (X). FIG. 5B, virus titers in BALB/c mice. FIG. 5C, weight loss of ICR mice after challenge with 150LD₅₀ A/PR/8 virus; vaccinated mice (filled circles); control mice (empty circles); survival of vaccinated (n=10) vs. AdC68rab.gp immune mice (n=10), p=0.01. FIG. 5D, survival curves of ICR mice after challenge with 150LD₅₀ of A/PR/8 virus; vaccinated mice (filled circles); control mice (empty circles); survival of vaccinated (n=10) vs. AdC68rab.gp immune mice (n=10), p=0.01. FIG. 5E, HE-stained sections of lungs harvested 5 days after challenge with 10LD₅₀ of A/PR/8 virus (100× magnification).

FIG. 6, protection of aged mice. Old and young C57Bl/6 mice were primed with AdC68M2e(3)-NP and 2 months later boosted with AdC6M2e(3)-NP. Three months later vaccinated and age-matched naïve control mice were challenged with 3LD₅₀ A/Fort Monmouth virus. Five days after challenge, virus titers in the lung were determined. Old vaccinated mice (n=8) vs. old control mice (n=10), p=0.3. Young vaccinated mice (n=15) verse young control mice (n=10), p=0.03.

FIGS. 7A-D, correlates of protection. FIG. 7A, protection of β2-microglobin knockout mice. The graph shows the survival curves of naïve and vaccinated (prime/boost regimen) mice upon challenge with 10LD₅₀ of A/PR/8 virus. Vaccinated mice (n=6) vs. naïve mice (n=6), p=0.2. FIG. 7B, protection by adoptive transfer of immune sera. C57Bl/6 mice were given 1.0 ml of sera harvested from C57Bl/6 mice vaccinated with AdC68M2e(3)-NP/AdC6M2e(3)-NP vectors. Naive mice received 1.0 ml of naïve sera, 24 hours later, mice were challenged with 10LD₅₀ of A/PR/8 virus. The graph shows the survival curves. Recipients of immune sera (n=10) vs. controls (n=10), p=0.02. FIG. 7C, frequencies of NP-specific CD8⁺ T cells measured 1 day before challenge from the blood of the same mice shown in (d) determined by tetramer staining. FIG. 7D, protection of AdC68NP vaccinated mice with or without immune sera transfer. The graph shows the survival curves. Groups of AdC68NP-immune C57Bl/6 mice (n=10 in each group) were injected with 1.0 ml of sera from C57Bl/6 mice prime-boosted with AdC68M2e(3)-NP/AdC6M2e(3)-NP. Another AdC68NP-vaccinated group was given 1.0 ml of naïve sera/mouse. The control group received 1.0 ml naïve serum/mouse. AdC68NP+ immune sera mice vs. naïve mice, p=0.01. AdC68NP vaccinated mice vs. naïve mice, p=0.09. AdC68NP/AdC6NP-vaccinated mice vs. naïve mice, p=0.2.

FIG. 8. Survival curves of naïve and vaccinated (prime/boost regimen) mice upon challenge with 10LD₅₀ of A/PR/8 virus.

### DETAILED DESCRIPTION

A universal influenza vaccine was designed to induce broadly cross-reactive immunity against current and future influenza A virus strains. The vaccine uses a vaccine platform that is uniquely suited to induce potent and sustained immune responses.

Experiments reported in the specific examples, below, demonstrate that a vaccine regimen based on sequential immunization with two serologically distinct chimpanzee-derived replication-defective adenovirus (Ad) vectors expressing the matrix-2 protein ectodomain (M2e) from 3 divergent strains of influenza A virus fused to the influenza virus nucleoprotein (NP) induces antibodies to M2e and virus-specific CD8⁺ T cells to NP. In pre-clinical mouse models, Ad vaccines expressing M2e and NP elicit robust NP-specific CD8⁺ T cell responses and moderate antibody responses to all 3 M2e sequences. Both antibody and CD8⁺ T cell responses can be enhanced by a second immunization with the Ad vector.

Inbred as well as outbred mice primed with the AdC68M2eNP vector or primed and then boosted with the AdC6M2eNP vector showed solid protection against challenge with different strains of influenza A virus. Vaccinated mice are protected against morbidity and mortality following challenge with high doses of different influenza virus strains. For example, protection was achieved against A/PR/8/34 virus from which the NP and one of the M2e sequences originated; however, protection was also achieved against A/Fort Monmouth/1/47, which carries an M2e sequence that is divergent from those expressed by the vaccines. In contrast, a vaccine based on a replication-defective adenovirus vector of chimpanzee serotype 68 expressing the nucleoprotein (NP) of influenza A/PR8 virus showed limited efficacy against A/PR8 infection in mice although the vaccine induced potent NP-specific CD8⁺ T cell responses.

### Fusion Polypeptides

Universal influenza vaccines described herein provide fusion polypeptides which comprise at least two components derived from at least two different influenza A strains. In some embodiments fusion polypeptides comprise (1) a first matrix protein ectodomain from a first strain of influenza A virus (M2e₁); and (2) a nucleoprotein (NP) from a second strain of influenza A virus. In some embodiments fusion polypeptides further comprise a second matrix protein ectodomain from a second strain of influenza A virus (M2e₂). In embodiments comprising two matrix protein ectodomains and a nucleoprotein, at least two of these three components are from different strains of influenza A virus. In other embodiments, all three components are from different strains of influenza A virus. The two (or three) components can be in any order.

In some embodiments fusion polypeptides comprise four components derived from at least two different influenza A strains: (1) a first matrix protein ectodomain from a first strain of influenza A virus (M2e₁); (2) a second matrix protein ectodomain from a second strain of influenza A virus (M2e₂); (3) a third matrix protein ectodomain from a third strain of influenza A virus (M2e₃); and (4) a nucleoprotein (NP) from a fourth strain of influenza A virus.

Suitable influenza A strains from which components of the fusion polypeptide can be derived include H1N1 (e.g., A/Puerto Rico/8/1934; A/Fort Monmouth/1/1947), H5N1 (e.g., A/Hong Kong/483/1997), H7N2 (e.g., A/Duck/Tasmania/277/2007), H1N2 (e.g., A/Swine/Korea/CY02/02), H2N2 (e.g., A/Leningrad/134/17/57), and H3N2 (e.g., A/New York/392/2004).

In some embodiments the first strain is an H1N1 strain. In some of these embodiments the H1N1 strain is A/Fort Monmouth/1/1947. In other embodiments the H1N1 strain is A/Puerto Rico/8/1934.

In some embodiments the first strain is an H5N1 strain. In some of these embodiments the H5N1 strain is A/Hong Kong/483/1997.

In some embodiments the first strain is an H7N2 strain. In some of these embodiments the H7N2 strain is A/Duck/Tasmania/277/2007.

In some embodiments the fourth strain is an H1N1 strain. In some embodiments both the first and the fourth strains are H1N1 strains and can be the same or different. In some of these embodiments the first H1N1 strain is A/Fort Monmouth/1/1947. In other embodiments the first H1N1 strain is A/Puerto Rico/8/1934.

In some embodiments the four components are ordered, from N to C terminus, M2e₁-M2e₂-M2e₃-NP. In some embodiments the NP and the M2e₁ are from A/Puerto Rico/8/1934; M2e₂ is from A/Hong Kong/483/1997; and the M2e₃ is from A/Duck/Tasmania/277/2007.

Although in specific examples below the M2e components from three strains are in the order H1N1-H5N1-H7N2, they an be used in any order.

### Nucleic Acid Molecules: Adenovirus Vectors

Nucleic acid molecules (either ribonucleic acid or deoxyribonucleic acid) encoding the fusion polypeptides can be constructed using standard recombinant nucleic acid techniques, for example as described in the examples, below.

Production, purification and quality control procedures for Ad vectors are well established.¹⁷ Ad vectors induce innate immune responses ameliorating the need for addition of adjuvants. They also induce very potent B and CD8⁺ T cell responses, which, due to low-level persistence of the vectors, are remarkably sustained.²⁹ Pre-existing neutralizing antibodies to common human serotypes of Ad viruses such as serotype which impact vaccine efficacy,¹⁷ can readily be avoided by the use of by serotypes from other species such as chimpanzees which typically neither circulate in humans nor cross-react with human serotypes.³⁰ In cases where prime-boost regimens are needed to achieve immune responses of sufficient potency, vectors based on distinct Ad serotypes are available.¹⁷ Ad viruses and Ad vectors have been used extensively in the clinic where they were well tolerated. They can be applied through a variety of routes including mucosal routes such as the airways³¹ or even orally upon encapsidation as was shown with vaccine to Ad viruses 4 and 7 used by the US military.³²

E1-deleted adenovirus vectors are well known in the art and are described, for example, in ref, 17. Preferably the adenovirus is a serotype from a species other than human, such as chimpanzee (e.g., AdC68 or AdC6). See also refs. 19 and 31.

### Vaccine Compositions: Methods of Immunizing

Universal influenza vaccines can be formulated using standard techniques and can comprise, in addition to an E1-deleted adenovirus vector encoding a fusion polypeptide, a pharmaceutically acceptable vehicle, such as phosphate-buffered saline (PBS) or other buffers, as well as other components such as antibacterial and antifungal agents, isotonic and absorption delaying agents, adjuvants, and the like. In some embodiments vaccine compositions are administered in combination with one or more other vaccines, including other influenza vaccines (e.g., seasonal vaccines). In some embodiments other influenza vaccines are peptide-based universal influenza vaccines (e.g., U.S. Pat. No. 7,354,589; and U.S. Pat. No. 7,527,798).

Universal influenza vaccines can be administered to individuals in need thereof to induce an immune response against strains of influenza A virus other than the strain(s) from which the vaccine components were derived. In some embodiments administration follows a “prime-boost” regimen, in which a second dose of a vaccine is provided some time after the first (e.g., 1, 2, 3, or 4 weeks or 1, 2, 3, or 4 months after the first dose). Typical dosage amounts range from 10⁷-10¹¹ virus particles. Methods of administration include, but are not limited to, mucosal (e.g., intranasal), intraperitoneal, intramuscular, intravenous, and oral administration. Immune responses can be assessed using suitable methods known in the art, including those taught in the specific examples, below.

Those skilled in the art will appreciate that there are numerous variations and permutations of the above described embodiments that fall within the scope of the appended claims.

### Example 1

### Material and Methods

### Adenovirus Vectors.

AdC68 and AdC6 vectors expressing the M2e(3)-NP chimeric protein were generated as follows: The 3 M2e encoding sequences with a signal peptide was synthesized by Integrated DNA Technologies (Coraville, Iowa) and cloned into pShuttle (Clontech, Mountain View, Calif.). The NP gene, upon deletion of the start codon, was cloned in frame downstream of the M2e sequences. Upon digestion with I-Ceu I and PI-Sce I, the fusion gene was cloned from pShuttle into the E1 domain of the molecular clones of AdC68 and AdC6, respectively. Recombinant Ad vectors (AdC68M2e(3)-NP and AdC6M2e(3)-NP) were rescued by transfection of plasmid DNA into HEK 293 cells. The Ad vectors were purified by cesium chloride density gradient centrifugation and virus particle (vp) content was determined by spectrophotometry at 260 nm. Vectors were titrated to determine infectious units (IU) and vector batches had vp to IU ratios below 200 and were cleared for endotoxin contamination. Other vectors encoding NP only or the glycoprotein of rabies virus (rab.gp), were generated and quality controlled using the same methods.

### Expression of the Vaccine Antigen.

HEK 293 cells were infected with 10-1000 vp per cell. 24 hours after infection Western blots were performed and membranes were blotted with a monoclonal antibody to M2e (14C2-S1-4.2).

### Influenza Virus.

Influenza viruses A/PR/8/34 and A/Fort Monmouth/1/47 were grown in the chorioallantoic fluid of embryonated chicken eggs and titrated in adult mice upon their intranasal infection to determine the mean lethal dose (LD₅₀).

### Mice.

Female C57Bl/6, BALB/e and ICR mice were purchased at 6-8 weeks of age from ACE Animals (Boyertown, Pa.). Female C57BL/6J mice (β2M^{-/-}, strain B6.129P-B2m^{tmIUnc}) were purchased at 6-8 weeks of age from Jackson Laboratory (Bar Harbor, Me.). All mice were housed in the Wistar Institute Animal Facility. C57Bl/6 mice were aged at the Animal Facility of the Wistar Institute and used once they were >20 months old. Animal procedures in this study were conducted in accordance with Institutional Animal Care and Use Committee guidelines.

### Immunization of Mice.

Groups of 6-15 mice were vaccinated with 1×10¹⁰ vp of the AdC68M2e(3)-NP vector given i.m. Two months later, some groups of mice were boosted with 1×10¹⁰ vp of the AdC6M2e(3)-NP vector given i.m.

### Challenge of Mice.

Two months after vaccination, mice were anesthetized and then challenged intranasally with either 10 or 150 LD₅₀ of influenza A/PR/8/34 virus or 3 or 10 LD₅₀ of influenza A/Fort Monmouth/1/47 virus, diluted in 30 μl PBS. Mice were weighed daily. They were euthanized if they lost in excess of 30% of their pre-challenge weight. In some experiments mice were euthanized 5 days after challenge.

### Virus Titration.

The assay was adopted from a previously published method³³ that was validated against the standard plaque assay. Lung tissue samples were excised from experimental mice, and their weight was recorded. After tissue samples were mechanically homogenized, RNAs were isolated by using TRIZOL® reagent (Invitrogen) and were resuspended in 50 μL of DEPC-treated water (Ambion). The RNA concentration of each sample was determined spectrophotometrically at an absorbance of 260 nm. From the entire 50 μL RNA solutions, cDNA was obtained using 100 μL reaction volumes with the manufacturer-specified component proportions of a High-Capacity cDNA Archive Kit (Applied Biosystems). Reactions were run on thermal cycler (Eppendorf MASTERCYCLER®) in one cycle at 25° C. for 10 min, 37° C. for 120 min, and 85° C. for 5 min. Concentrations of cDNA were standardized to 50 ng/5 mL, and influenza A/PR8 cDNA was used to create a standard curve by serial dilution, ranging from 4 ng/5 mL to 0.0064 ng/5 mL. Viral cDNA was quantified using a TaqMan® real-time PCR assay on an ABI PRISM® 7000 Sequence Detector. The primers for viral cDNA quantification were specific to the influenza A matrix protein gene (MP), which were MP sense (5′-AAGACC AATCCTGTCACCTCTGA-3% SEQ ID NO:5) and MP antisense (5′-CAAAGCGTCTACGCTGCAGTCC-3′; SEQ ID NO:6). The reporter probe was a TAQMAN® TAMARA™ (Applied Biosystems) of sequence [6-FAM]-5′-TTTGTGTTCACGCTCACCGTT (SEQ ID NO:7)-3′-[TAMARA]. The cDNA samples were quantified in triplicate. Each reaction totaled 25 μl and included 12.5 μL TaqMan® Universal PCR Master Mix, 5 pmol reporter probe, 22 pmol MP sense primer, 22 pmol MP antisense primer, and 5 μL (50 ng) cDNA sample template. Reactions were run at 50° C. for 2 min, 95° C. for 10 min, and then cycled 40 times between 95° C. for 15 s and 60° C. for 1 min. In analyzing the spectral curves, the cycle threshold was defined just above the emission baseline to stay within the exponential amplification phase of the PCR. Viral copy numbers were normalized with the original tissue sample masses, and calculated based on the molar mass of influenza A/PR8 genome.

### Antibody Titers to M2e.

Antibody responses specific to M2e were measured from sera of individual mice by a cellular ELISA that was modified from a previously published procedure.¹⁸ We cloned the three frill-length M2 sequences from which the M2e sequences of the vaccines had originated, into lentivirus vectors. Lentivirus was rescued in 293T cells and used to infect HeLa cells to generate stable cell lines that express full length M2. A control cell line was generated by infection of 2931 cells with empty lentivirus. These cell lines were used as immunosorbents in an ELISA as described.¹⁸ The assay was standardized with a purified antibody that recognizes all of the 3 M2e sequences (manuscript in preparation).

### Intracellular Cytokine Staining (ICS).

Frequencies of NP-specific IFN-γ-producing CD8⁺ cell were determined following vaccination at different time points from blood as described.³⁴ Samples were analyzed using an EPICS XL (Beckman-Coulter, Brea, Calif.). FlowJo 7.1.1 software (Tree Star Inc., Ashland, Oreg.) was used for post-acquisition analysis. Frequencies of NP-specific CD8⁺ T cells are shown as IFN-γ⁺ CD8⁺ over all CD8⁺ cells.

### Tetramer Staining.

Lymphocytes were isolated from lung, blood, and spleen of individual mice before or 5 days after challenge. Cells were stained with an APC-labeled MHC class I NP peptide tetramer (ASNENTE™; SEQ ID NO:8, Tetramer Core Facility, Emory University, Ga.), in combination with an anti-CD8a-PerCP-Cy5.5 antibody (BD Biosciences, San Jose, Calif.) for 1 h at 4° C. Flow cytometry was performed with the Beckman-Coulter XL, (Beckman-Coulter, Brea, Calif.) at The Wistar Institute Flow Cytometry Core Facility and data were analyzed with FlowJo 7.1.1 (Tree Star Inc., Ashland, Oreg.).

### Histology.

Lungs were perfused with 1% FBS supplemented PBS and the lobes were gently inflated with 200 μL of a 10% formalin solution through a 30 g needle. The inflated lung samples were submerged in 10% formalin for tissue fixation for 24 hours at 4° C. Formalin-fixed lung samples were paraffin-embedded and sectioned at 4 μm and mounted on glass slides. Sections were stained with H&E and two random sections of each lung sample were examined. Histopathological changes were examined by an investigator, who was unaware of the samples' origin. Lung pathology was scored as follows: 1—no observable pathology; 2—perivascular infiltrates, 3—perivascular and interstitial infiltrates affecting <20% of the lobe section; 4—perivascular and interstitial infiltrates affecting 20-50% of the lobe section; 5—perivascular and interstitial infiltrates affecting >50% of the lobe section.

### Statistical Analyses.

Immune responses, pathology grade, as well as virus titers were analyzed using samples from individual mice. Results are shows as mean±standard deviation (SD). Significance of differences between groups was determined by one-tailed Student's t-test or ANOVA. Differences between pathology scores were analyzed by Wilcoxon two sample test. The statistical significance of protection of vaccinated groups compared to the control group was determined using Fisher's exact test.

### Example 2

### Transgene Product Expression

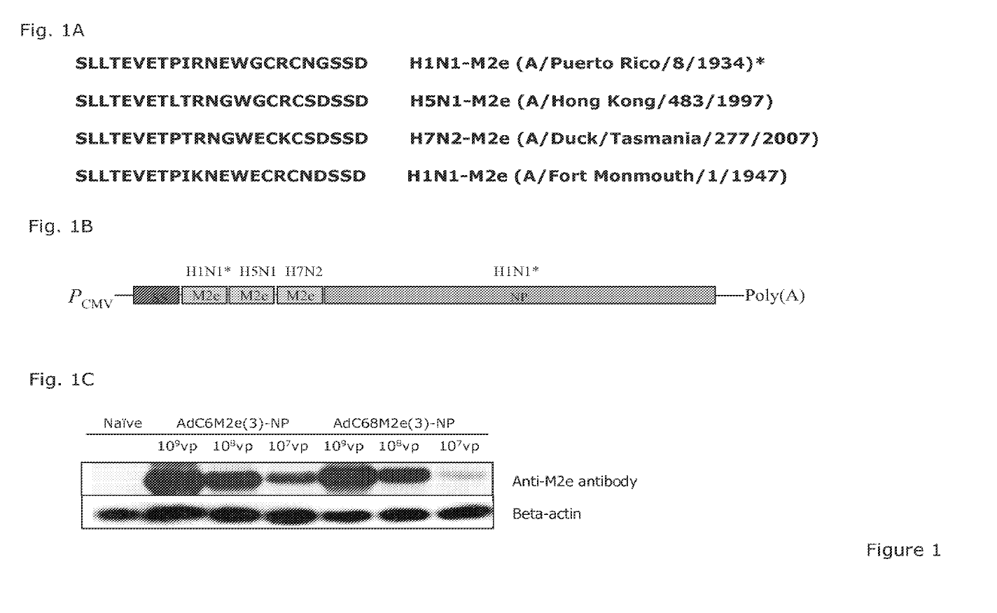

The M2e(3)-NP chimeric gene encodes the M2e of A/PR/8, an H1N1 virus, a pathogenic H5N1 virus that evolved in 1997, and an avian H7N2 strain isolated in 2007 (FIG. 1A). The 3 M2e sequences were combined with the full-length NP sequence. Linker sequences, encoding three alanine residues, were inserted between each gene and a signal sequence from HSV-1 glycoprotein D was placed upstream of the chimeric gene (FIG. 1B). Western Blotting showed that AdC68M2e(3)-NP and AdC6M2e(3)-NP express comparable levels of the chimeric protein in vitro using a monoclonal antibody to M2e termed 14C2-S1-4.2¹⁸ as shown in FIG. 1C or an antibody to NP (not shown).

### Example 3

### Antibody Responses to M2e

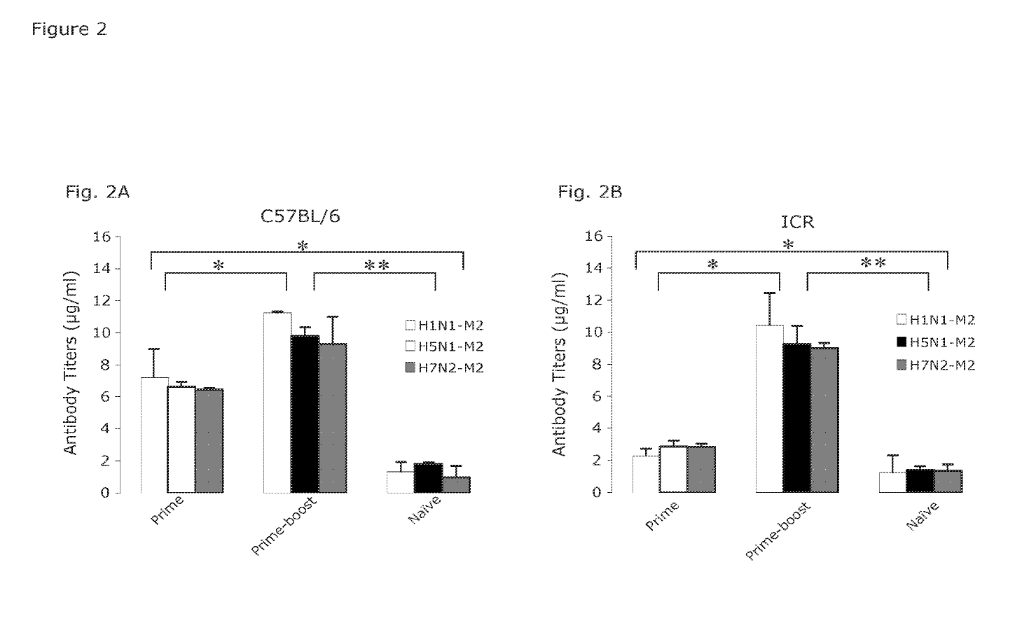

Groups of young C57Bl/6 mice were vaccinated with 1×10¹⁰ vp of AdC68M2e(3)-NP, some of them were boosted 2 months later with 1×10¹⁰ vp of AdC6M2e(3)-NP. Sera were harvested from individual mice 5 weeks after the boost, and together with naïve control sera or, in separate experiments, sera from mice vaccinated with vectors expressing the rabies virus glycoprotein (rab.gp), tested for antibodies to M2e on the different M2 transfected or sham-transfected HeLa cell lines (FIG. 2A). Antibody titers were comparable upon testing on the 3 cell lines and increased after the boost. Sera from mice vaccinated with the control vectors showed background reactivity similar to that of sera from naïve mice (e.g., average antibody titers to M2e in naive ICR mice, 1.2 μg/ml; average antibody titers in ICR mice after an AdCrab.gp prime boost regimen: 0.99 μg/ml). To ensure that the vaccine induced a response in genetically distinct strains of mice, outbred ICR mice were tested using the same vaccine regimens. Antibody titers achieved after priming were lower than those in C57Bl/6 mice, but comparable after the boost (FIG. 2B).

### Example 4

NP-specific CD8⁺ T cell responses

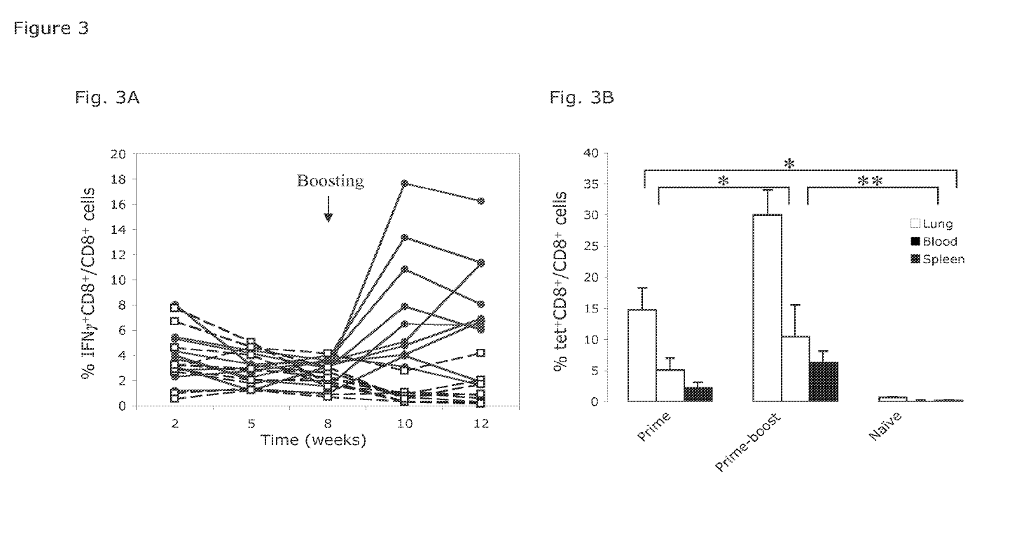

Vaccine-induced CD8⁺ cell responses to NP were tested at different time points after vaccination by intracellular cytokine staining for IFN-γ (FIG. 3A). After priming with AdC68M2e(3)-NP, all of the mice developed detectable frequencies of NP-specific CD8⁺ cells in blood, which gradually declined. A booster immunization with AdC6M2e(3)-NP given 2 months after priming affected an increase in circulating NP-specific CD8⁺ T cells. Mice were euthanized 4 months after priming and frequencies of NP-specific CD8⁺ T cells were determined from lymphocytes isolated from blood, spleens and lungs of individual mice (FIG. 3B). Frequencies were higher in mice that had received the prime-boost regimens; frequencies were highest in lungs and lowest in spleens. Higher frequencies in peripheral tissues that primary attract effector/effector memory cells that in lymphatic tissues such as spleen is typical for Ad vector induced cell responses as has been described previously.¹⁹

### Example 5

### Protective Immunity

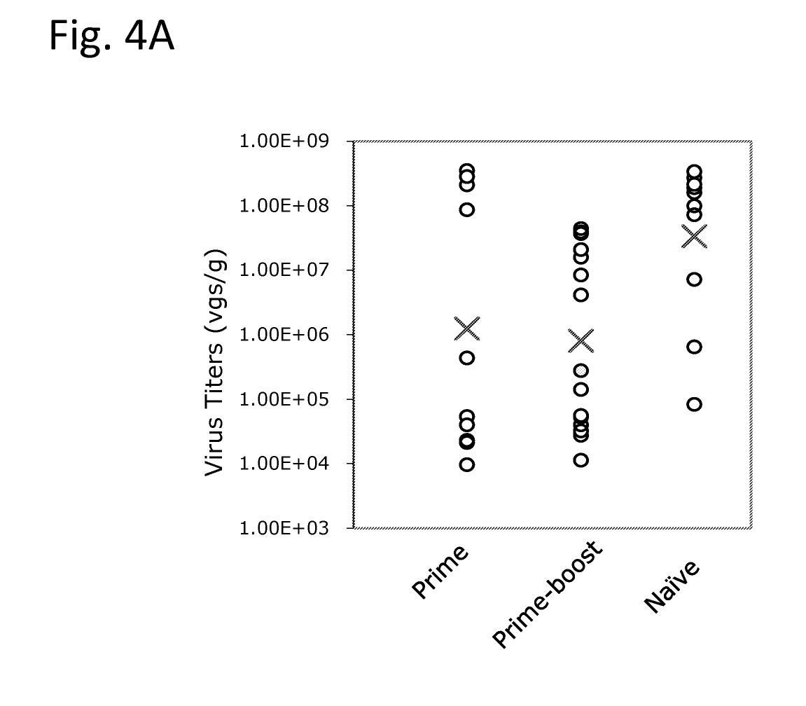

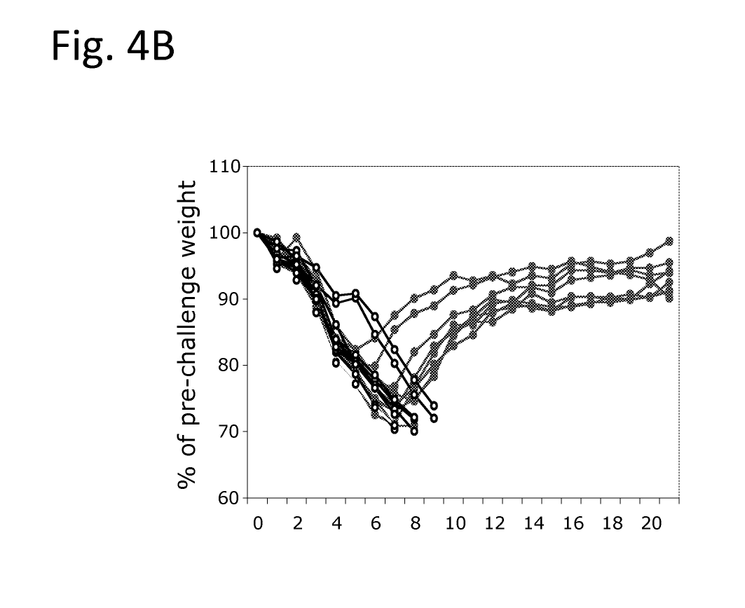

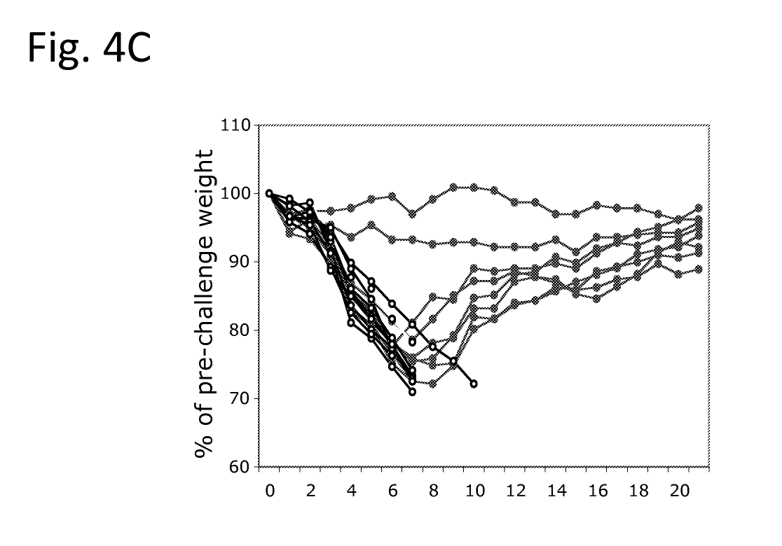

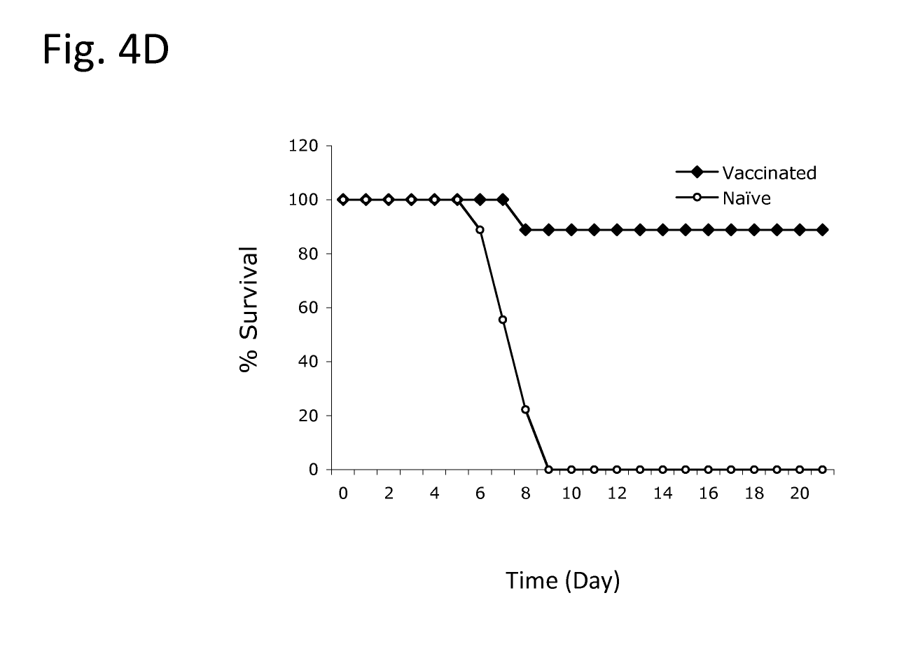

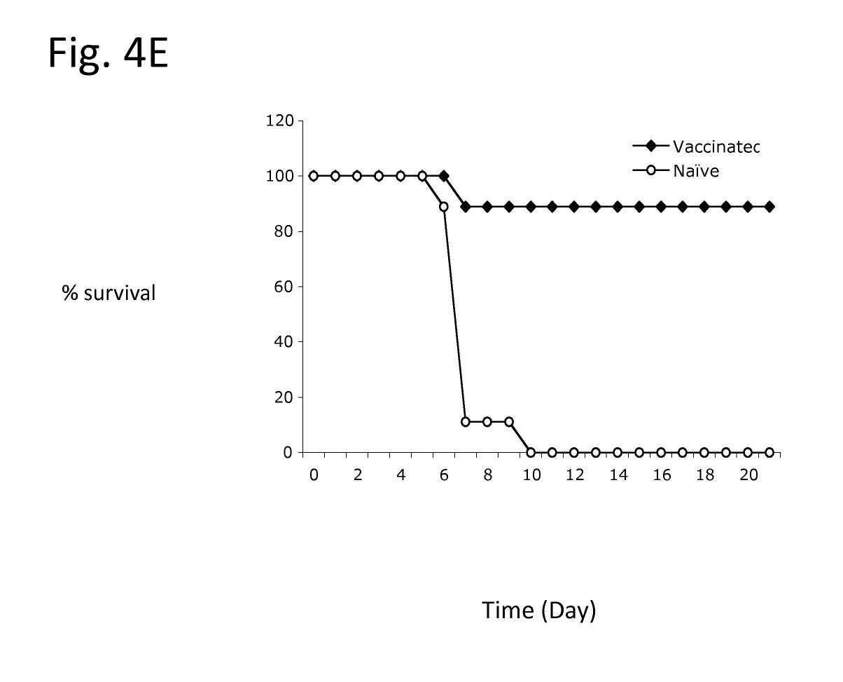

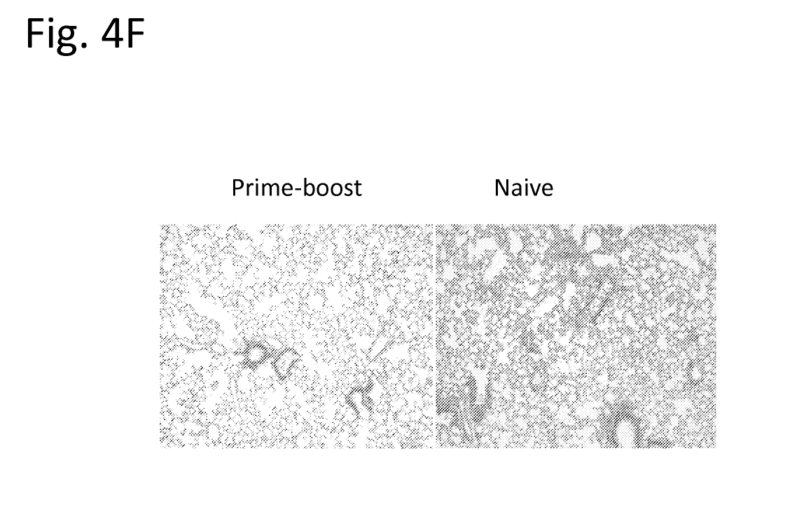

C57Bl/6 mice were vaccinated and then infected with 10LD₅₀ of A/PR/8 virus. Lung virus titers were measured from the right inferior lobe of the lungs of individual mice 5 days after challenge (FIG. 4A). This time point was chosen for the following reasons. Influenza virus replication can be detected rapidly within 48 hours in lungs of mice, depending on the dose of challenge virus in mice that are able to fend off the infection titers then start to decline around day 7.²⁰ Vaccines that induce neutralizing antibodies would be expected to decrease virus titers from the onset while vaccine such as ours that induces immune mechanisms directed against infected cells would be expected to act with a delay. Mice that had been primed with AdC68M2e(3)-NP showed a reduction in mean titers but this did not reach significance compared to control mice (p=0.2). A significant reduction in lung viral titers was achieved upon prime-boosting (p=0.0003). To ensure that the reduction in viral titers resulted in a clinical benefit, the experiment was repeated with the prime-boost regimen and mice were challenged with 10LD₅₀ of A/PR/8 or A/Fort Monmouth virus. Vaccinated, naïve or sham-vaccinated mice lost weight after challenge. Weight loss of vaccinated mice peaked by days 6-8 after challenge and then mice began to gain weight and by 21 days after challenge most mice had returned to their pre-challenge weight. Naïve or sham-vaccinated control mice continued to lose weight after challenge till they died or required euthanasia (FIGS. 4B, 4C). Upon challenge with either virus strain, 90% of the vaccinated mice survived while all of the control mice died (FIGS. 4D, 4E). Lung lobes harvested 5 days after challenge were stained with H&E and analyzed for signs of inflammation using the scoring system described in the material and method section. Most of the unvaccinated mice had perivascular infiltrates in their lungs and half of them had interstitial infiltrates with an average pathology score of 2.35 (FIG. 4F). Pathology was less pronounced in mice that had received the prime-boost vaccination, and the average pathology score of their lungs was 1.85 (p=0.04). We infected mice through the experiments at 4 months after priming. Those that received a second does of vaccine were boosted at 2 months after priming and then challenged 2 months later. This protocol allowed us to prime and challenge mice together thus reducing experimental variability. One could make the argument that differences in the time interval between vaccination and challenge may have biased the results. We therefore in additional experiments tested, mice that only received one dose of the AdC68M2e(3)-NP vaccine at 2 months after vaccination and protection was comparable to that observed in mice challenged at 4 months after immunization.

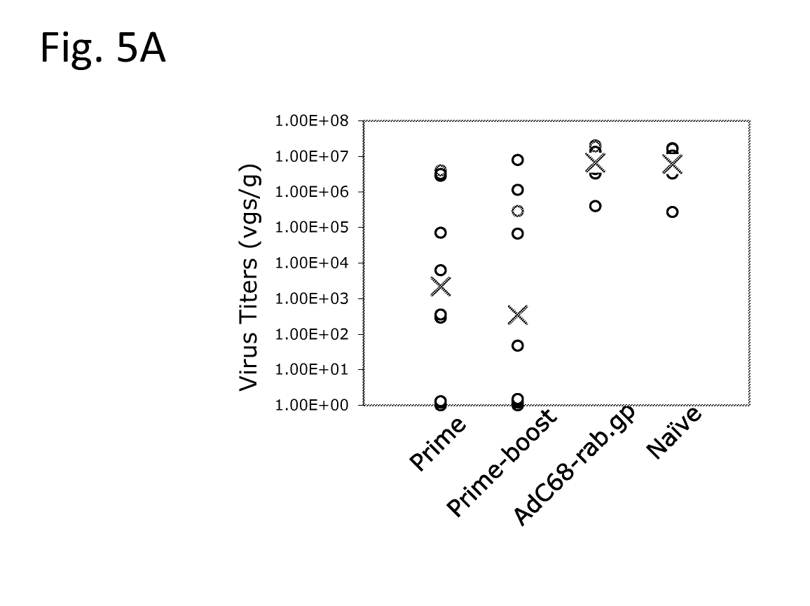

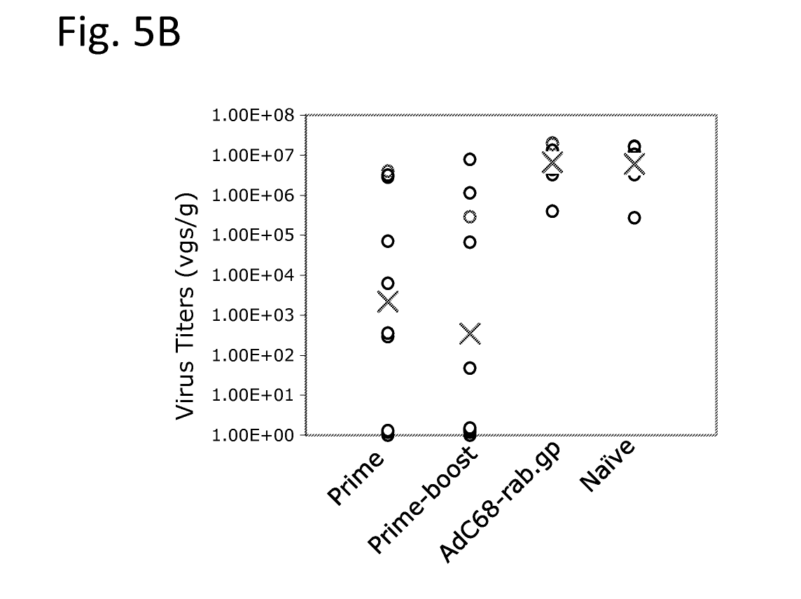

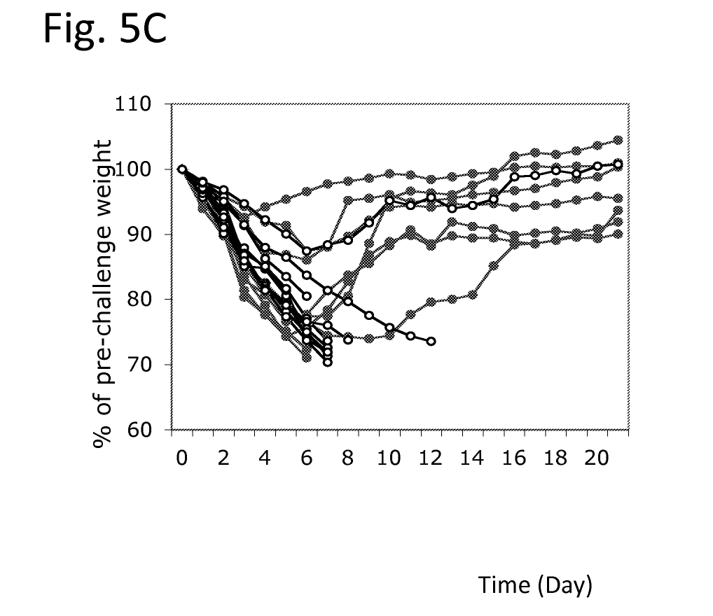

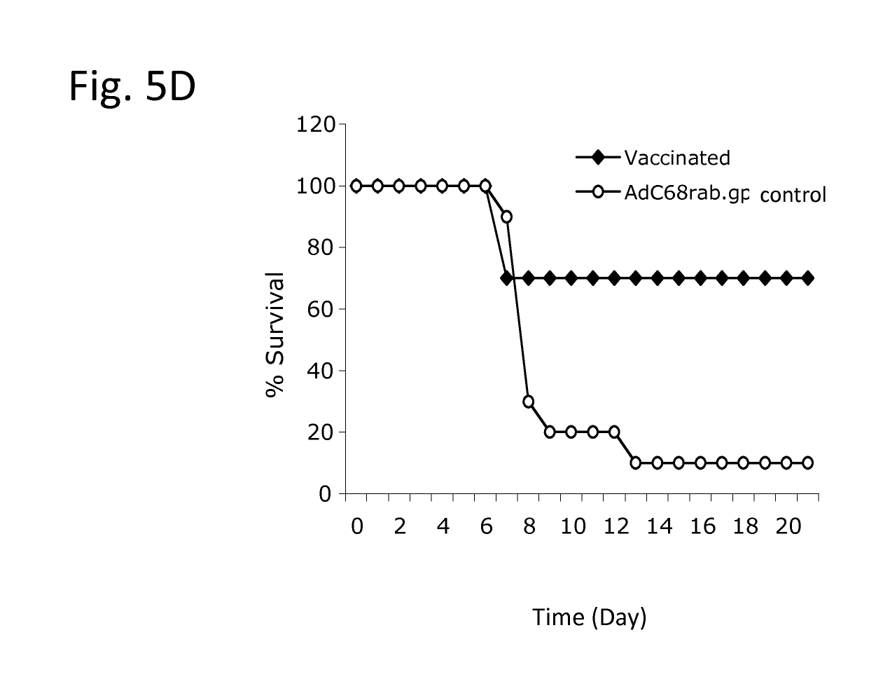

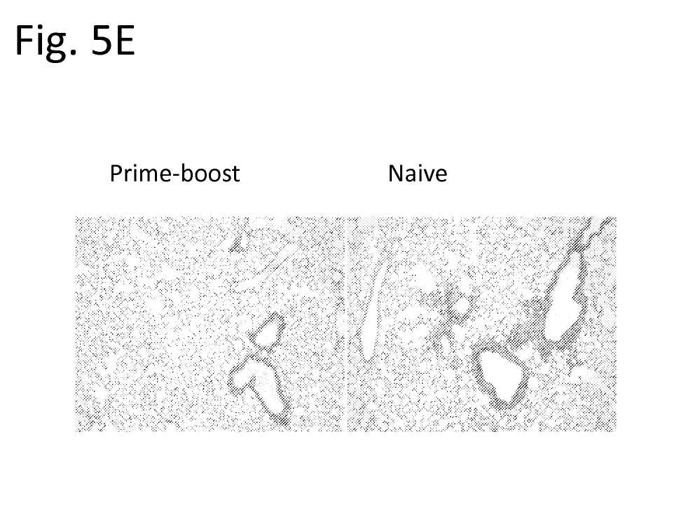

The experiment was repeated with ICR and BALB/c mice. Mice were challenged 4 months after priming or 2 months after the boost with 10LD₅₀ of A/PR/8 virus. By day 5 after challenge lung virus titers were significantly reduced in ICR mice that were primed (p=0.0002) or primed and boosted (p=0.0003) (FIG. 5A). In 30% of primed mice and 50% of mice that received the prime-boost regimen virus had been cleared completely from their lungs while all of the mice of the two control groups had titers in excess of 10⁵ genome copies. Similar results were obtained with BALB/c mice (FIG. 5B).

Next vaccinated ICR mice were challenged with an increased dose of 150LD₅₀ of A/PR/8 virus (FIGS. 5C, 5D). In spite of this very severe challenge, which killed 90% of the control mice, 70% of the vaccinated mice survived. Histological analyses (FIG. 5E) of lung section of ICR mice conducted 5 days after challenge revealed a significant reduction in inflammation in vaccinated mice with an average pathology score of 2.7 in naïve mice and 1.9 in vaccinated mice (p=0.009).

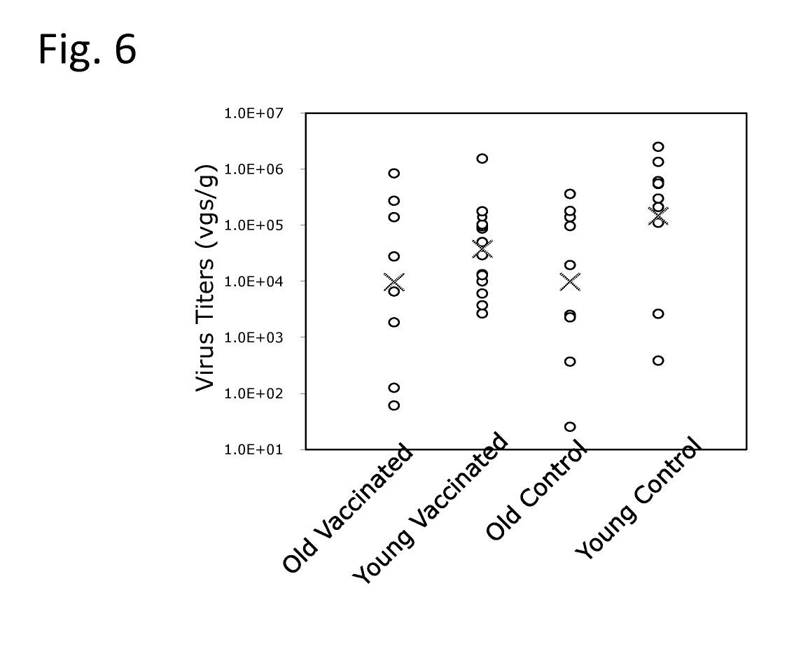

Influenza, viruses cause death mainly in the aged and commercially available vaccines are commonly poorly immunogenic in this population.²¹ To test if the AdM2e(3)-NP vectors induce protection in aged mice, we immunized a group of 20 months old C57Bl/6 mice with AdC68M2e(3)-NP and boosted them 2 months later with AdC6M2e(3)-NP. Mice were challenged 3 months after the boost with 3LD₅₀ of A/Fort Monmouth virus and viral titers were determined from lungs 5 days later. Groups of young mice were tested in parallel. Antibody responses to M2e were comparable in old and young vaccinated mice although old naive mice had slightly higher background titers (average antibody titers to M2e in old mice: 17 μg/ml; average background titers in old mice: 6 μg/ml). Frequencies of NP-specific CD8⁺ T cells were higher in aged mice at the time of challenge (% specific CD8⁺ cells/all CD8⁺ T cells: old mice: 10.2%; young mice: 5.9%, p=0.03). By 5 days after challenge, lung virus titers in young vaccinated mice were significantly below those of young naïve mice, while such a difference was not seen in aged mice, although they had on average lower titers compared to the young (FIG. 6). In addition, young vaccinated mice showed reduced weight loss following challenge compared to young naïve mice, which again was not seen for the aged mice indicating that the vaccines although they induce immune responses in aged mice, nevertheless, lacked efficacy in this population.

### Example 6

### Immune Correlates of Protection

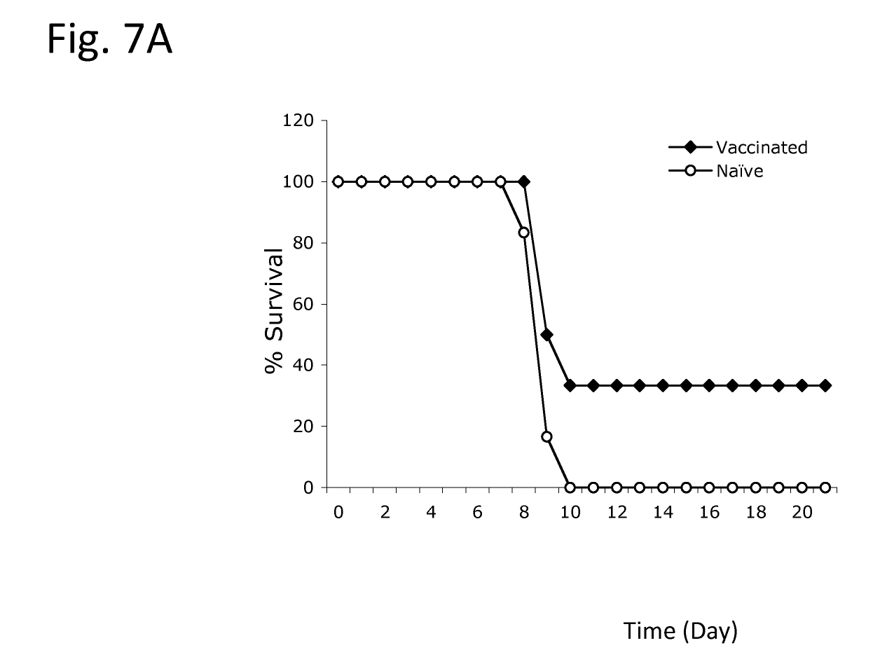

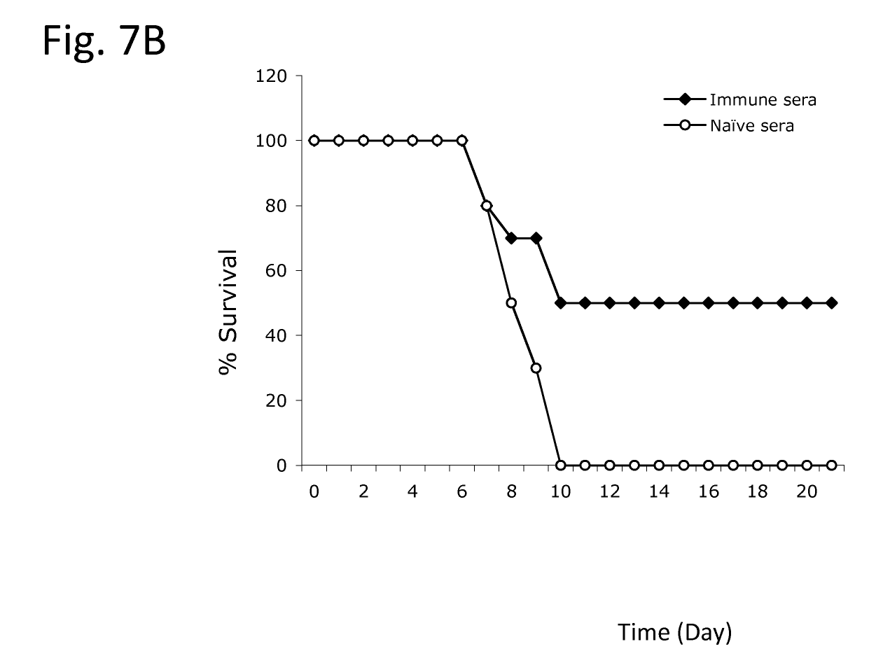

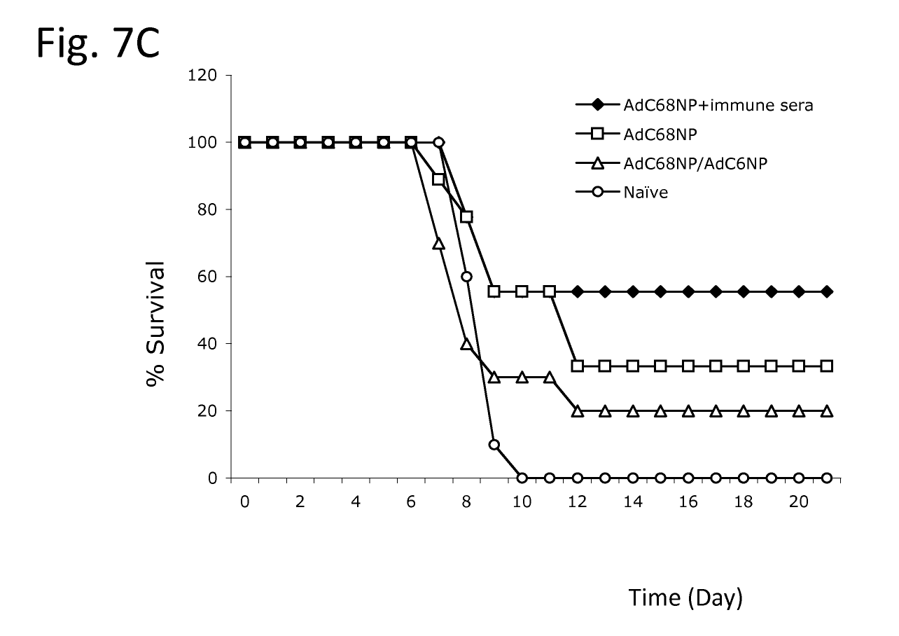

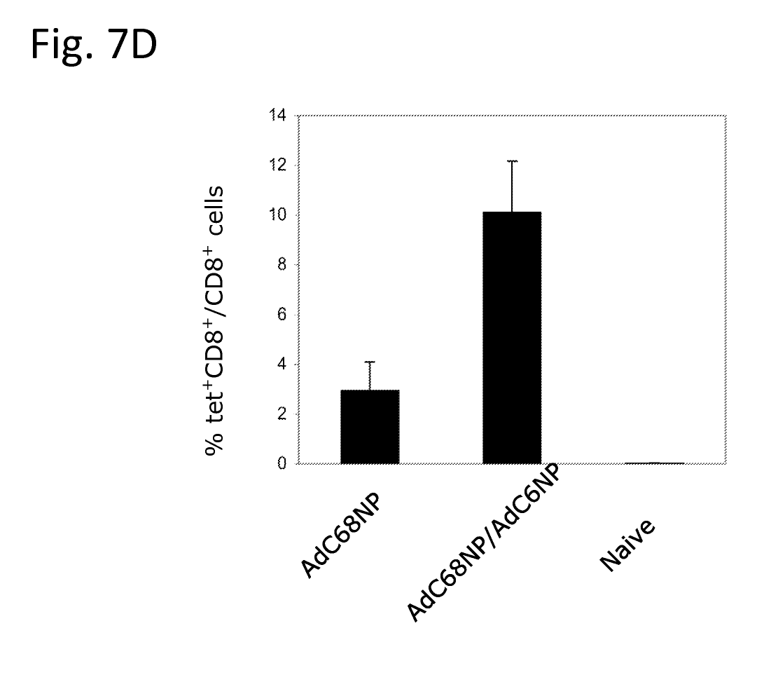

To elucidate the immune mechanisms that contribute to protection in vaccinated mice, the vaccines were tested in 132-microglobin knockout mice, which lack CD8⁺ cells. Mice received a prime-boost regimen or were left naïve. Antibody titers measured at 5 weeks after the boost were comparable to those achieved in wild-type C57Bl/6 mice (mean titer of 10.7 μg of antibodies to M2e/ml). Upon challenge of mice with 10 LD₅₀ A/PR/8, only 33.3% ( 2/6) of vaccinated mice survived while all of the naïve mice succumbed the infection (FIG. 7A). This difference was not significant suggesting that antibodies alone failed to provide protection against severe challenge.

In a second experiment, the role of antibodies was assessed by adoptive transfer studies. Donor C57Bl/6 mice received the prime-boost regimen with the AdCM2e(3)-NP vectors and 1 ml of their pooled sera, which at the time of harvest contained 14.3 μg M2e-specific antibodies/ml, was transferred to naive C57Bl/6 recipients, which were challenged 24 hours later with 10LD₅₀ of A/PR/8. Control mice received sera from naïve donors. Transfer of M2e immune sera protected 50% of the recipients, while all of the mice injected with the control sera died following challenge (FIG. 7B).

To further assess correlates of protection, mice were vaccinated with an AdC68 vector expressing NP only (AdC68NP) or they were primed with AdC68NP and then boosted 2 months later with AdC6NP. Frequencies of NP-specific CD8⁺ T cells were measured from blood 2 months later (FIG. 7C) and were found to be comparable to those achieved with the AdC68M2e(3)-NP vaccine or a prime-boost regimen with the two heterologous Ad vectors. It should also be noted that AdNP-vaccinated mice developed antibodies to NP. Two months after vaccination, one group of AdC68NP-vaccinated mice received 1.0 ml of sera obtained from C57Bl/6 mice that had received the prime-boost regimen with the AdM2e(3)-NP vectors, the other group received 1.0 ml of sera from naïve mice. Mice were challenged 24 hours later with 10LD₅₀ of A/PR/8 virus. All of the control animals died, while 33.3% of AdC68NP vaccinated mice transferred with naïve serum survived (FIG. 7D). This degree of survival did not reach statistical significance. Survival was significant at 55.6% in AdC68-NP-vaccinated mice that received the immune serum again suggesting that vaccine-induced protection against high dose challenge requires both antibodies to M2e and CD8⁺ T cells to NP. Enhancing NP-specific CD8⁺ T cells by booster immunization did not result in increased protection, but mice rather showed a trend towards accelerated death potentially suggesting that a potent NP-specific CD8⁺ T cell response may exacerbate disease. In addition, these results show that a combination of T cells and antibodies to NP does not suffice for protection by that antibodies to M2e are essential.

### Example 7

Comparison of immunization with M2e and NP vs M2e or NP Alone

The combination of M2e and NP together in a viral vector achieves greater protection effect against influenza virus infection than using M2e or NP alone. The results from the following experiment provide support for the statement.

Four groups of female C57Bl/6 mice at 6-8 weeks of age were vaccinated using a prime and boost regimen, with a resting period of 2 months between priming and boosting. All immunizations were given intramuscularly. There were 10 mice in each experimental group and 9 mice in the control group. The first group of mice were vaccinated with AdC-M2e(3)-NP, the second group with AdC-M2e(3), and the third group with AdC-NP. The last group of mice was a control group and vaccinated with vectors containing an irrelevant transgene (rab.gp, rabies virus G protein). The Adenoviral vectors used were chimpanzee serotypes C68 for priming and C6 for boosting. The table below shows the immunizations used for each group.

| Group | Prime | Boost |
|---|---|---|
| Group 1 | AdC68-M2e(3)-NP | AdC6-M2e(3)-NP |
| Group 2 | AdC68-M2e(3) | AdC6-M2e(3) |
| Group 3 | AdC68-NP | AdC6-NP |
| Group 4 | AdC68-rab.gp | AdC6-rab.gp |

Two months after boosting, all mice were challenged with Influenza A/PR/834 virus at the dose of 10 LD₅₀ given intranasally. Mice were weighed right before and also daily after given the challenge Influenza, virus. Mice were observed for 21 days, and the numbers of mice survived from each group were recorded. When the weight loss exceeded 30% from the pre-challenge weight, mice were euthanized.

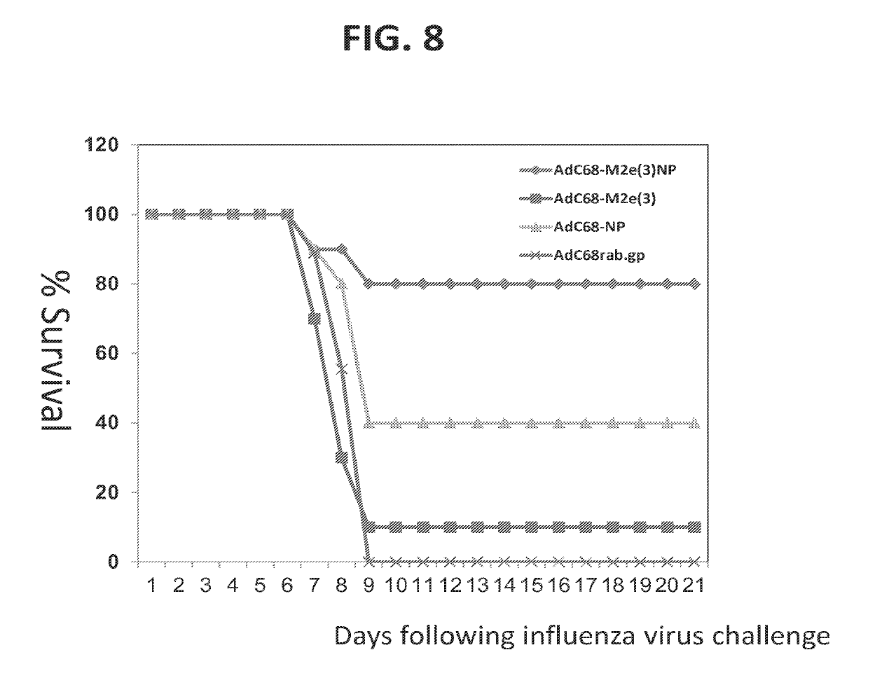

The results are shown in FIG. 8. Mice immunized with the adenovirus vector encoding M2e(3)-NP had higher survival rates than mice immunized with either M2e(3) or NP alone. Surprisingly, the percent survival of the M2e(3)-NP-immunized mice was greater than the combination of the survival rates of the M2e(3)- and NP-immunized mice.

### REFERENCES

1. Thompson, WW, Comanor, L, Shay, DK (2006) Epidemiology of seasonal influenza: use of surveillance data and statistical models to estimate the burden of disease. J Infect Dis 194: S82-91.
2. Nichol, KL, Mendelman, PM, Mallon, KP, Jackson, L A, Gorse, GJ, Beishe. R B et al. (1999) Effectiveness of live, attenuated intranasal influenza virus vaccine in healthy, working adults: a randomized controlled trial. JAMA 282: 137-144.
3. Fan, J, Liang, X, Horton, M S, Perry, HC, Citron, MP, Heidecker, G J et al. (2004) Preclinical study of influenza virus A M2 peptide conjugate vaccines in mice, ferrets, and rhesus monkeys. Vaccine 22: 2993-3003.
4. Neirynck, S. Deroo, I, Saelens, X, Vanlandschoot, P, Jou, WM, Fiefs, W (1999) A universal influenza A vaccine based on the extracellular domain of the M2 protein. Nat Med 5: 1157-1163.
5. Carragher, DM, Kaminski, DA, Moquin, A, Hanson, L, and Randall, TD (2008) A novel role for non-neutralizing antibodies against nucleoprotein in facilitating resistance to influenza virus. J Immunol 1181: 4168-4176.
6. Epstein, SL, Kong, WP, Misplon, JA, Lo, CY, Tumpey, TM, Xu, L et al. 2005. Protection against multiple influenza. A subtypes by vaccination with highly conserved nucleoprotein. Vaccine 23: 5404-5410.
7. Yewdell, JW, Bennink, JR, Smith, GL, Moss, B (1985) Influenza A virus nucleoprotein is a major target antigen for cross-reactive anti-influenza A virus cytotoxic T lymphocytes. Proc Natl Acad Sci USA 82: 1785-1789.
8. McMichael, AJ, Michie, C A, Gotch, FM, Smith, GL, Moss, B (1986) Recognition of influenza A virus nucleoprotein by human cytotoxic T lymphocytes. J Gen Virol 67: 719-726.
Epstein, SL (2006) Prior H1N1 influenza infection and susceptibility of Cleveland Family Study participants during the H2N2 pandemic of 1957: an experiment of nature. J Infect Dis 193: 49-53.
10. Mozdzanowska, K (2003) Induction of influenza type A virus-specific resistance by immunization of mice with a synthetic multiple antigenic peptide vaccine that contains ectodomains of matrix protein 2. Vaccine 21: 2616-2626.
11. Huleatt, JW, Nakaar, V, Desai, P. Huang, Y, Hewitt, D, Jacobs, A et al. (2008) Potent immunogenicity and efficacy of a universal influenza vaccine candidate comprising a recombinant fusion protein linking influenza M2e to the TLR5 ligand flagellin. Vaccine 26: 201-214.
12. Zhao, G, Lin, Y, Du, L, Guan, J, Sun, S. Sui, H et al. (2010) An M2e-based multiple antigenic peptide vaccine protects mice from lethal challenge with divergent H5N1 influenza viruses. Virol J. 7: 9.
13. Schotsaert, M, De Filette, M, Fiers, W, Saelens, X (2009) Universal M2 ectodomain-based influenza A vaccines: preclinical and clinical developments. Expert Rev Vaccines 8: 499-508.
14. http://clinicaltrials.gov/ct2/show/NCT00993083
15. Jefferson, TO, Rivetti, D, Di Pietrantonj, C, Rivetti, A, Demicheli, V (2007) Vaccines for preventing influenza in healthy adults. Cochrane Database Syst Rev 18: CD001269.
16. Smith, S, Demicheli, V, Di Pietrantonj, C, Harnden, A R, Jefferson, T, Matheson, N J et al. (2006) Vaccines for preventing influenza in healthy children. Cochrane Database Syst Rev 1: CD004879.
17. Tatsis, N. Ertl, UC (2004) Adenoviruses as vaccine vectors. Mol Ther 10: 616-629.
18. Zhang, M, Zharikova, D, Mozdzanowska, K, Otvos, L, Gerhard, W (2006) Fine specificity and sequence of antibodies directed against the ectodomain of matrix protein 2 of influenza A virus. Mol Immunol 43: 2195-2206.
19. Tatsis, N, Lin, SW, Harris-McCoy, K, Garber, DA, Feinberg, MB, Ertl, HC (2007) Multiple immunizations with adenovirus and MVA vectors improve CD8+ T cell functionality and mucosal homing. Virology 367: 156-167.
20. Taylor, RM (1941) Experimental infection with influenza A virus in mice: the increase in intrapulmonary virus after inoculation and the influence of various factors thereon. J Exp Med 73: 43-55.
21. Nichol, KL, Margolis, KL, Wouremna, J, von Sternberg; T (1996) Effectiveness of influenza vaccine in the elderly. Gerontology 42: 274-279.
22. Ulmer, JB, Fa, TM, Deck, RR, Friedman, A, Guan, L, DeWitt, C. Liu, X et al. (1998) Protective CD4+ and CD8+ T cells against influenza virus induced by vaccination with nucleoprotein DNA. J Virol 72: 5648-5653.
23. Lawson, CM, Bennink, JR, Restito, NP, Yewdell, JW, Murphy, BR (1994) Primary pulmonary cytotoxic T lymphocytes induced by immunization with a vaccinia virus recombinant expressing influenza A virus nucleoprotein peptide do not protect mice against challenge. J Virol 68: 3505-3511.
24. Heinen, PP, Rijsewijk, FA, de Boer-Luijtze, EA, Bianchi, AT (2002) Vaccination of pigs with a DNA construct expressing an influenza virus M2-nucleoprotein fusion protein exacerbates disease after challenge with influenza A virus. J Gen Virol 83: 1851-1859.
25. Lamb, RA, Zebedee, SL, Richardson, CD (1985) Influenza virus M2 protein is an integral membrane protein expressed on the infected-cell surface. Cell 40: 627-633.
26. Feng, J, Zhang, M, Mozdzanowska, K, Zharikova, D, Hoff, H, Wunner, W et al. (2006) Influenza A virus infection engenders a poor antibody response against the ectodomain of matrix protein 2. Virol J 3: 102.
27. Liu, W, Zou, P. Ding, J, Lu, Y, Chen, YH (2005) Sequence comparison between the extracellular domain of M2 protein human and avian influenza A virus provides new information for bivalent influenza vaccine design. Microbes Infect 7: 171-177.
28. Zharikova, D, Mozdzanowska, K, Feng, J, Zhang, M, and Gerhard, W (2005) Influenza type A virus escape mutants emerge in vivo in the presence of antibodies to the ectodomain of matrix protein 2. J Virol 79: 6644-6654.
29. Tatsis, N, Fitzgerald, JC, Reyes-Sandoval, A, Harris-McCoy, KC, Hensley, SE, Zhou, D et al. (2007) Adenoviral vectors persist in vivo and maintain activated CD8+ T cells: implications for their use as vaccines. Blood 110: 1916-1923.
30. Xiang, Z, Li, Y, Cun, A, Yang, W, Ellenherg, S. Switzer, W M et al. (2006) Chimpanzee adenovirus antibodies in humans, sub-Saharan Africa. Emerg Infect Dis 12: 1596-9.
31. Xiang, ZQ, Gao, GP, Reyes-Sandoval, A, Li, Y, Wilson, JM, Ertl, HC (2003) Oral vaccination of mice with adenoviral vectors is not impaired by preexisting immunity to the vaccine carrier. J Virol 77: 10780-10789.
32. Lyons, A, Longfield, J, Kuschner, R, Straight, Seriwatana, J et al. (2008) A double-blind, placebo-controlled study of the safety and immunogenicity of live, oral type 4 and type 7 adenovirus vaccines in adults. Vaccine 26: 2890-2898.
33. DiMenna, L, Latimer, B, Parzych, E, Haut, LH, Töpfer, K. Abdulla, S et al. (2010). Augmentation of primary influenza A virus specific CD8⁺ cell responses in aged mice through blockade of an immunoinhibitory pathway. J Immunol 184: 5475-5484.
34. Fitzgerald, JC, Gao, GP, Reyes-Sandoval, A, Pavlakis, GN, Xiang, ZQ, Wlazlo, A P et al. (2003) A simian replication-defective adenoviral recombinant vaccine to HIV-1 gag. J Immunol 170: 1416-1422.

1. A fusion polypeptide comprising four components:
a first matrix protein ectodomain from a first strain of influenza A virus (M2e₁); and
a second matrix protein ectodomain from a second strain of influenza A virus (M2e₂);
a third matrix protein ectodomain from a third strain of influenza A virus (M2e₃); and
a nucleoprotein (NP) from a fourth strain of influenza A virus,
wherein at least two of the first, second, third, and fourth strains are different strains.

2. A fusion polypeptide comprising:
a first matrix protein ectodomain from a first strain of influenza A virus (M2e₁); and
a nucleoprotein (NP) from a different strain of influenza A virus.

3. The fusion polypeptide of claim 2 further comprising a second matrix protein ectodomain from a second strain of influenza A virus (M2e₂).

4. The fusion polypeptide of claim 1 wherein the four components are ordered, from N to C terminus, M2e₁-M2e₂-M2e₃-NP.

5. The fusion polypeptide of claim 1, wherein the first strain is an H1N1 strain.

6. The fusion polypeptide of claim 1, wherein the first strain is an H5N1 strain.

7. The fusion polypeptide of claim 1 wherein the first strain is an H7N2 strain.

8. The fusion polypeptide of claim 1 wherein the fourth strain is an H1N1 strain.

9. The fusion polypeptide of claim 1 wherein the first and fourth strains are the same.

10. A nucleic acid molecule encoding the fusion polypeptide of claim 1.

11. An E1-deleted adenovirus vector comprising the nucleic acid molecule 10.

12. The E1-deleted adenovirus vector of claim 11 which is derived from a chimpanzee serotype.

13. The E1-deleted adenovirus vector of claim 12 wherein the chimpanzee serotype is selected from the group consisting of C68 and C6.

14. A method of inducing an immune response against two or more strains of influenza A virus, comprising a first administration of the E1-deleted adenovirus vector of claim 11 to an individual in need thereof.

15. The method of claim 14 further comprising a second administration of the E1-deleted adenovirus vector.

16. The method of claim 14 wherein the administration is selected from the group consisting of mucosal, oral, intramuscular, intravenous, and intraperitoneal administration.

17-20. (canceled)
